(19) United States
(12) Patent Application Publication
(10) Pub. No.: US 2014/0025129 A1
(43) Pub. Date: Jan. 23, 2014

(54) MEDICAL DEVICE WITH PROTOCOL LOCALIZATION

(71) Applicants: Martin Abbenhouse, Kirkland, WA (US); David R. Christie, Woodinville, WA (US); Mitchell A. Smith, Sammamish, WA (US); Daniel Conan Perreault, Everett, WA (US)

(72) Inventors: Martin Abbenhouse, Kirkland, WA (US); David R. Christie, Woodinville, WA (US); Mitchell A. Smith, Sammamish, WA (US); Daniel Conan Perreault, Everett, WA (US)

(73) Assignee: PHYSIO-CONTROL, INC., Redmond, WA (US)

(21) Appl. No.: 13/552,552

(22) Filed: Jul. 18, 2012

(51) Int. Cl. A61N 1/39

(52) U.S. Cl. CPC A61N 1/39
USPC 607/5

(57) ABSTRACT

An external medical device can include a housing, an energy storage module within the housing for storing an electrical charge, and a defibrillation port for guiding via electrodes the stored electrical charge to a person. The device can also include a user interface to deliver prompts to a user during a defibrillation session and a locale detector in the housing to determine a present locale of the device. The prompts delivered by the user interface to the user during the defibrillation session can be in accordance with a protocol that is selected based on the present locale.

### FIELD

This invention generally relates to medical devices, such as external defibrillators.

### BACKGROUND

In humans, the heart beats to sustain life. In normal operation, it pumps blood through the various parts of the body. More particularly, the various chamber of the heart contract and expand in a periodic and coordinated fashion, which causes the blood to be pumped regularly. More specifically, the right atrium sends deoxygenated blood into the right ventricle. The right ventricle pumps the blood to the lungs, where it becomes oxygenated, and from where it returns to the left atrium. The left atrium pumps the oxygenated blood to the left ventricle. The left ventricle, then, expels the blood, forcing it to circulate to the various parts of the body.

The heart chambers pump because of the heart's electrical control system. More particularly, the sinoatrial (SA) node generates an electrical impulse, which generates further electrical signals. These further signals cause the above-described contractions of the various chambers in the heart, in the correct sequence. The electrical pattern created by the sinoatrial (SA) node is called a sinus rhythm.

Sometimes, however, the electrical control system of the heart malfunctions, which can cause the heart to beat irregularly, or not at all. The cardiac rhythm is then generally called an arrhythmia. Arrhythmias may be caused by electrical activity from locations in the heart other than the SA node. Some types of arrhythmia may result in inadequate blood flow, thus reducing the amount of blood pumped to the various parts of the body. Some arrhythmias may even result in a Sudden Cardiac Arrest (SCA). In a SCA, the heart fails to pump blood effectively, and, if not treated, death can occur. In fact, it is estimated that SCA results in more than 250,000 deaths per year in the United States alone. Further, a SCA may result from a condition other than an arrhythmia.

One type of arrhythmia associated with SCA is known as Ventricular Fibrillation (VF). VF is a type of malfunction where the ventricles make rapid, uncoordinated movements, instead of the normal contractions. When that happens, the heart does not pump enough blood to deliver enough oxygen to the vital organs. The person's condition will deteriorate rapidly and, if not reversed in time, they will die soon, e.g. within ten minutes.

Ventricular Fibrillation can often be reversed using a life-saving device called a defibrillator. A defibrillator, if applied properly, can administer an electrical shock to the heart. The shock may terminate the VF, thus giving the heart the opportunity to resume pumping blood. If VF is not terminated, the shock may be repeated, often at escalating energies.

A challenge with defibrillation is that the electrical shock must be administered very soon after the onset of VF. There is not much time: the survival rate of persons suffering from VF decreases by about 10% for each minute the administration of a defibrillation shock is delayed. After about 10 minutes the rate of survival for SCA victims averages less than 2%.

The challenge of defibrillating early after the onset of VF is being met in a number of ways. First, for some people who are considered to be at a higher risk of VF or other heart arrhythmias, an Implantable Cardioverter Defibrillator (ICD) can be implanted surgically. An ICD can monitor the person's heart, and administer an electrical shock as needed. As such, an ICD reduces the need to have the higher-risk person be monitored constantly by medical personnel.

Regardless, VF can occur unpredictably, even to a person who is not considered at risk. As such, VF can be experienced by many people who lack the benefit of ICD therapy. When VF occurs to a person who does not have an ICD, they collapse, because blood flow has stopped. They should receive therapy quickly.

For a VF victim without an ICD, a different type of defibrillator can be used, which is called an external defibrillator. External defibrillators have been made portable, so they can be brought to a potential VF victim quickly enough to revive them.

During VF, the person's condition deteriorates, because the blood is not flowing to the brain, heart, lungs, and other organs. Blood flow must be restored, if resuscitation attempts are to be successful.

Cardiopulmonary Resuscitation (CPR) is one method of forcing blood flow in a person experiencing cardiac arrest. In addition, CPR is the primary recommended treatment for some patients with some kinds of non-VF cardiac arrest, such as asystole and pulseless electrical activity (PEA). CPR is a combination of techniques that include chest compressions to force blood circulation, and rescue breathing to force respiration.

Properly administered CPR provides oxygenated blood to critical organs of a person in cardiac arrest, thereby minimizing the deterioration that would otherwise occur. As such, CPR can be beneficial for persons experiencing VF, because it slows the deterioration that would otherwise occur while a defibrillator is being retrieved. Indeed, for patients with an extended down-time, survival rates are higher if CPR is administered prior to defibrillation.

Advanced medical devices can actually coach a rescuer who performs CPR. For example, a medical device can issue instructions, and even prompts, for the rescuer to perform CPR more effectively.

### BRIEF SUMMARY

The present description gives instances of devices, systems, software and methods, the use of which may help overcome problems and limitations of the prior art.

In certain embodiments, an external medical device may include a housing, an energy storage module within the housing for storing an electrical charge, and a defibrillation port for guiding via electrodes the stored electrical charge to a person. The device may also include a user interface structured to deliver prompts to a user during a defibrillation session and a locale detector in the housing structured to determine a present locale of the device. The prompts delivered by the user interface to the user during the defibrillation session may be in accordance with a protocol that is selected based on the present locale.

An advantage over the prior art is that an external medical device in accordance with the disclosed technology can deliver the prompts in accordance with a protocol associated with the present location of the device, regardless of whether the device has been recently moved to a different location. Also, because the protocol may specify a particular language in which the user interface is to deliver the prompts to the user during a defibrillation session, for example, the device can deliver the prompts in a language that may be most suitable for the present location of the device.

These and other features and advantages of this description will become more readily apparent from the following Detailed Description, which proceeds with reference to the drawings, in which:

### BRIEF DESCRIPTION OF THE DRAWINGS

FIG. 1 is a diagram of a scene where an external defibrillator is used to save the life of a person according to embodiments.

FIG. 2 is a table listing two main types of the external defibrillator shown in FIG. 1, and who they might be used by.

FIG. 3 is a functional block diagram showing components of an external defibrillator, such as the one shown in FIG. 1, which is made according to embodiments.

FIG. 4 is a functional block diagram showing components of another external defibrillator, such as the one shown in FIG. 1, which is made according to certain embodiments.

FIG. 5 is a functional block diagram showing components of another external defibrillator, such as the one shown in FIG. 1, which is made according to certain embodiments.

FIG. 6A is a diagram showing an external defibrillator prompting a user to provide an indication of the present locale according to embodiments.

FIG. 6B is a diagram showing the user of FIG. 6A providing an indication of the present locale to the external defibrillator according to embodiments.

FIG. 6C is a diagram showing the external defibrillator of FIG. 6B prompting the user to provide a confirmation of a particular language according to embodiments.

FIG. 6D is a diagram showing the user of FIG. 6C providing a confirmation of the language to the external defibrillator according to embodiments.

FIG. 7A is a diagram showing a user crying out for help in the vicinity of an external defibrillator according to embodiments.

FIG. 7B is a diagram showing the external defibrillator of FIG. 7A prompting the user to provide a confirmation of a determined present locale according to embodiments.

FIG. 7C is a diagram showing the user of FIG. 7B providing a confirmation of the present locale to the external defibrillator according to embodiments.

FIG. 7D is a diagram showing the external defibrillator of FIG. 7C beginning to provide the user with instructions for using the external defibrillator according to embodiments.

FIG. 8 is a flowchart for illustrating methods of an external defibrillator delivering prompts in accordance with a selected protocol according to embodiments.

FIG. 9 is a flowchart for illustrating methods of an external defibrillator setting a protocol based on a determination of the present locale according to embodiments.

FIG. 10 is a flowchart for illustrating methods of an external defibrillator switching away from a current protocol for user prompts according to embodiments.

### DETAILED DESCRIPTION

As has been mentioned, the present description is about medical devices, methods of operating such medical devices, and a programmed processor to control such medical devices for controlling enabling features of the medical device based on protocol localization.

Embodiments are now described in more detail.

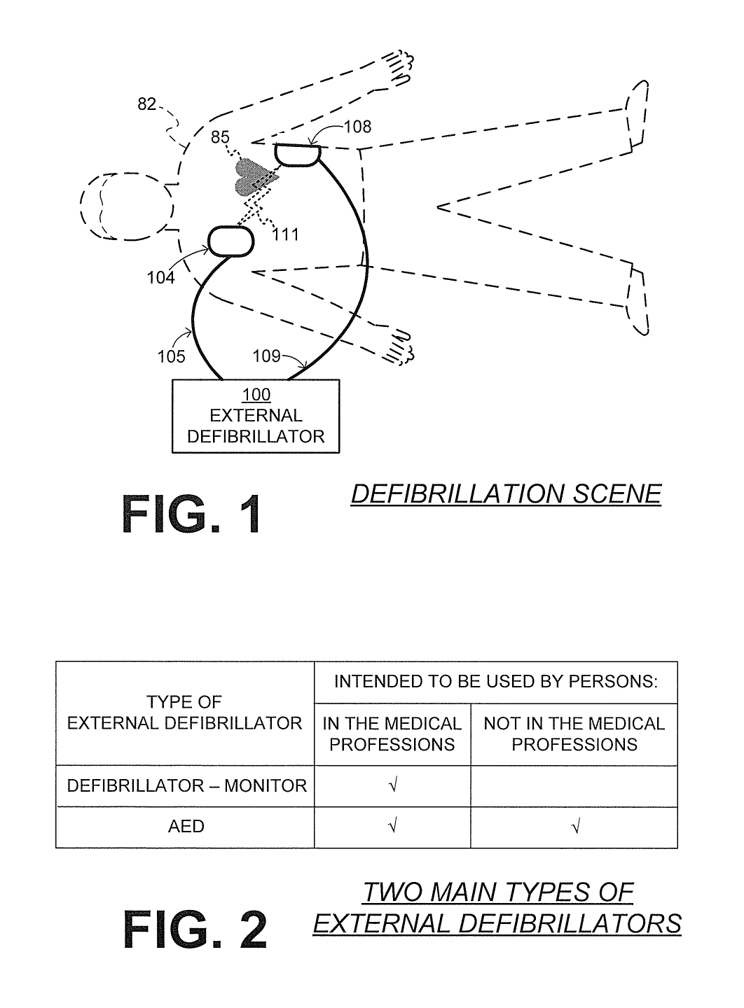

FIG. 1 is a diagram of a defibrillation scene. A person 82 is lying on their back. Person 82 could be a patient in a hospital, or someone found unconscious, and then turned to be on their back. Person 82 is experiencing a condition in their heart 85, which could be Ventricular Fibrillation (VF).

A portable external defibrillator 100 has been brought close to person 82. At least two defibrillation electrodes 104, 108 are usually provided with external defibrillator 100, and are sometimes called electrodes 104, 108. Electrodes 104, 108 are coupled with external defibrillator 100 via respective electrode leads 105, 109. A rescuer (not shown) has attached electrodes 104, 108 to the skin of person 82. Defibrillator 100 is administering, via electrodes 104, 108, a brief, strong electric pulse 111 through the body of person 82. Pulse 111, also known as a defibrillation shock, goes also through heart 85, in an attempt to restart it, for saving the life of person 82.

Defibrillator 100 can be one of different types, each with different sets of features and capabilities. The set of capabilities of defibrillator 100 is determined by planning who would use it, and what training they would be likely to have. Examples are now described.

FIG. 2 is a table listing two main types of external defibrillators, and who they are primarily intended to be used by. A first type of defibrillator 100 is generally called a defibrillator-monitor, because it is typically formed as a single unit in combination with a patient monitor. A defibrillator-monitor is sometimes called monitor-defibrillator. A defibrillator-monitor is intended to be used by persons in the medical professions, such as doctors, nurses, paramedics, emergency medical technicians, etc. Such a defibrillator-monitor is intended to be used in a pre-hospital or hospital scenario.

As a defibrillator, the device can be one of different varieties, or even versatile enough to be able to switch among different modes that individually correspond to the varieties. One variety is that of an automated defibrillator, which can determine whether a shock is needed and, if so, charge to a predetermined energy level and instruct the user to administer the shock. Another variety is that of a manual defibrillator, where the user determines the need and controls administering the shock.

As a patient monitor, the device has features additional to what is minimally needed for mere operation as a defibrillator. These features can be for monitoring physiological indicators of a person in an emergency scenario. These physiological indicators are typically monitored as signals. For example, these signals can include a person's full ECG (electrocardiogram) signals, or impedance between two electrodes. Additionally, these signals can be about the person's temperature, non-invasive blood pressure (NIBP), arterial oxygen saturation/pulse oximetry (SpO2), the concentration or partial pressure of carbon dioxide in the respiratory gases, which is also known as capnography, and so on. These signals can be further stored and/or transmitted as patient data.

A second type of external defibrillator 100 is generally called an AED, which stands for “Automated External Defibrillator”. An AED typically makes the shock/no shock determination by itself, automatically. Indeed, it can sense enough physiological conditions of the person 82 via only the shown defibrillation electrodes 104, 108 of FIG. 1. In its present embodiments, an AED can either administer the shock automatically, or instruct the user to do so, e.g. by pushing a button. Being of a much simpler construction, an AED typically costs much less than a defibrillator-monitor. As such, it makes sense for a hospital, for example, to deploy AEDs at its various floors, in case the more expensive defibrillator-monitor is more critically being deployed at an Intensive Care Unit, and so on.

AEDs, however, can also be used by people who are not in the medical profession. More particularly, an AED can be used by many professional first responders, such as policemen, firemen, etc. Even a person with only first-aid training can use one. And AEDs increasingly can supply instructions to whoever is using them.

AEDs are thus particularly useful, because it is so critical to respond quickly, when a person suffers from VF. Indeed, the people who will first reach the VF sufferer may not be in the medical professions.

Increasing awareness has resulted in AEDs being deployed in public or semi-public spaces, so that even a member of the public can use one, if they have obtained first aid and CPR/AED training on their own initiative. This way, defibrillation can be administered soon enough after the onset of VF, to hopefully be effective in rescuing the person.

There are additional types of external defibrillators, which are not listed in FIG. 2. For example, a hybrid defibrillator can have aspects of an AED, and also of a defibrillator-monitor. A usual such aspect is additional ECG monitoring capability.

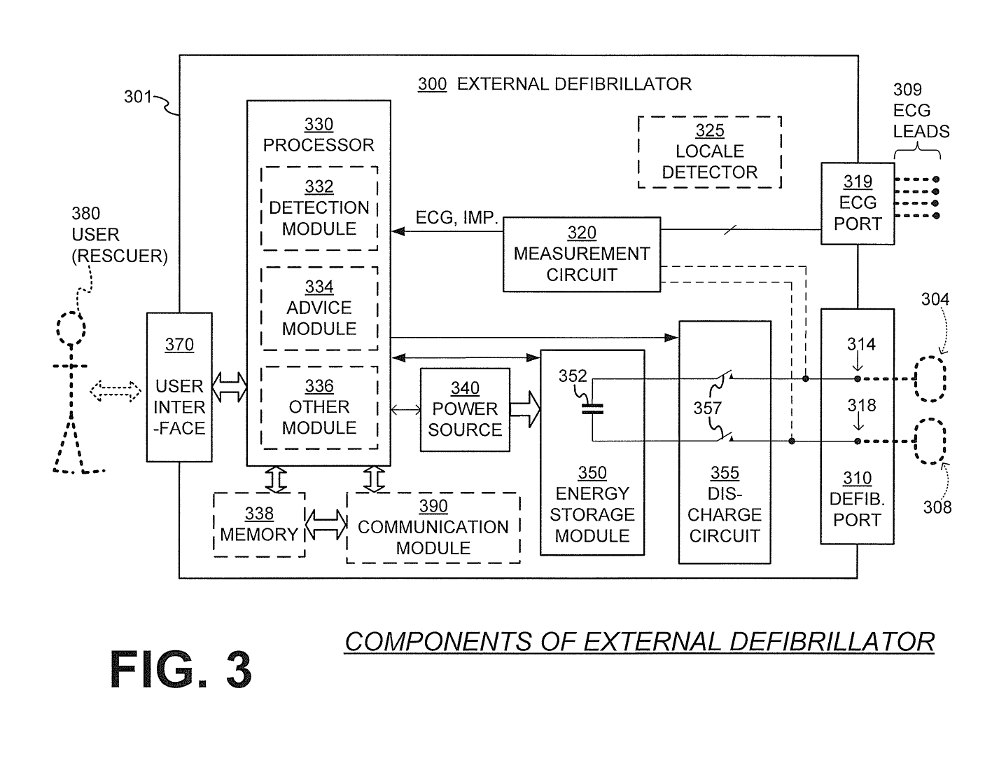

FIG. 3 is a diagram showing components of an external defibrillator 300 made according to embodiments. These components can be, for example, in external defibrillator 100 of FIG. 1. Plus, these components of FIG. 3 can be provided in a housing 301, which is also known as casing 301.

External defibrillator 300 is intended for use by a user 380, who would be the rescuer. Defibrillator 300 typically includes a defibrillation port 310, such as a socket in housing 301. Defibrillation port 310 includes nodes 314, 318. Defibrillation electrodes 304, 308, which can be similar to electrodes 104, 108, can be plugged in defibrillation port 310, so as to make electrical contact with nodes 314, 318, respectively. It is also possible that electrodes can be connected continuously to defibrillation port 310, etc. Either way, defibrillation port 310 can be used for guiding via electrodes to person 82 an electrical charge that has been stored in defibrillator 300, as will be seen later in this document.

If defibrillator 300 is actually a defibrillator-monitor, as was described with reference to FIG. 2, then it will typically also have an ECG port 319 in housing 301, for plugging in ECG leads 309. ECG leads 309 can help sense an ECG signal, e.g. a 12-lead signal, or from a different number of leads. Moreover, a defibrillator-monitor could have additional ports (not shown), and a locale detector 325 structured to determine a present locale of the defibrillator 300.

The locale detector 325 may determine the present locale based on one or more inputs received by the defibrillator 300. Alternatively or in addition to the locale detector 325, the defibrillator 300 may include a locale selector structured to select a present locale of the defibrillator 300. The defibrillator 300 may cause prompts, such as voice prompts, to be delivered to a user in accordance with a protocol that is selected based on the present locale of the defibrillator 300.

Defibrillator 300 also includes a measurement circuit 320. Measurement circuit 320 receives physiological signals from ECG port 319, and also from other ports, if provided. These physiological signals are sensed, and information about them is rendered by circuit 320 as data, or other signals, etc.

If defibrillator 300 is actually an AED, it may lack ECG port 319. Measurement circuit 320 can obtain physiological signals through nodes 314, 318 instead, when defibrillation electrodes 304, 308 are attached to person 82. In these cases, a person's ECG signal can be sensed as a voltage difference between electrodes 304, 308. Plus, impedance between electrodes 304, 308 can be sensed for detecting, among other things, whether these electrodes 304, 308 have been inadvertently disconnected from the person.

Defibrillator 300 also includes a processor 330. Processor 330 may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 can be considered to have a number of modules. One such module can be a detection module 332, which senses outputs of measurement circuit 320. Detection module 332 can include a VF detector. Thus, the person's sensed ECG can be used to determine whether the person is experiencing VF.

Another such module in processor 330 can be an advice module 334, which arrives at advice based on outputs of detection module 332. Advice module 334 can include a Shock Advisory Algorithm, implement decision rules, and so on. The advice can be to shock, to not shock, to administer other forms of therapy, and so on. If the advice is to shock, some external defibrillator embodiments merely report that to the user, and prompt them to do it. Other embodiments further execute the advice, by administering the shock. If the advice is to administer CPR, defibrillator 300 may further issue prompts for it, and so on.

Processor 330 can include additional modules, such as module 336, for other functions. In addition, if other component 325 is indeed provided, it may be operated in part by processor 330, etc.

Defibrillator 300 optionally further includes a memory 338, which can work together with processor 330. Memory 338 may be implemented in any number of ways. Such ways include, by way of example and not of limitation, nonvolatile memories (NVM), read-only memories (ROM), random access memories (RAM), any combination of these, and so on. Memory 338, if provided, can include programs for processor 330, and so on. The programs can be operational for the inherent needs of processor 330, and can also include protocols and ways that decisions can be made by advice module 334. In addition, memory 338 can store prompts for user 380, etc. Moreover, memory 338 can store patient data.

Defibrillator 300 may also include a power source 340. To enable portability of defibrillator 300, power source 340 typically includes a battery. Such a battery is typically implemented as a battery pack, which can be rechargeable or not. Sometimes, a combination is used, of rechargeable and non-rechargeable battery packs. Other embodiments of power source 340 can include AC power override, for where AC power will be available, and so on. In some embodiments, power source 340 is controlled by processor 330.

Defibrillator 300 additionally includes an energy storage module 350. Module 350 is where some electrical energy is stored, when preparing it for sudden discharge to administer a shock. Module 350 can be charged from power source 340 to the right amount of energy, as controlled by processor 330. In typical implementations, module 350 includes one or more capacitors 352, and so on.

Defibrillator 300 moreover includes a discharge circuit 355. Circuit 355 can be controlled to permit the energy stored in module 350 to be discharged to nodes 314, 318, and thus also to defibrillation electrodes 304, 308. Circuit 355 can include one or more switches 357. Those can be made in a number of ways, such as by an H-bridge, and so on.

Defibrillator 300 further includes a user interface 370 for user 380. User interface 370 can be made in any number of ways. For example, interface 370 may include a screen, to display what is detected and measured, provide visual feedback to the rescuer for their resuscitation attempts, and so on. Interface 370 may also include a speaker, to issue voice prompts, etc. Interface 370 may additionally include various controls, such as pushbuttons, keyboards, and so on. In addition, discharge circuit 355 can be controlled by processor 330, or directly by user 380 via user interface 370, and so on.

Defibrillator 300 can optionally include other components. For example, a communication module 390 may be provided for communicating with other machines. Such communication can be performed wirelessly, or via wire, or by infrared communication, and so on. This way, data can be communicated, such as patient data, incident information, therapy attempted, CPR performance, and so on.

A feature of a defibrillator can be CPR-prompting. Prompts are issued to the user, visual or by sound, so that the user can administer CPR. Examples are taught in U.S. Pat. No. 6,334,070 and No. 6,356,785.

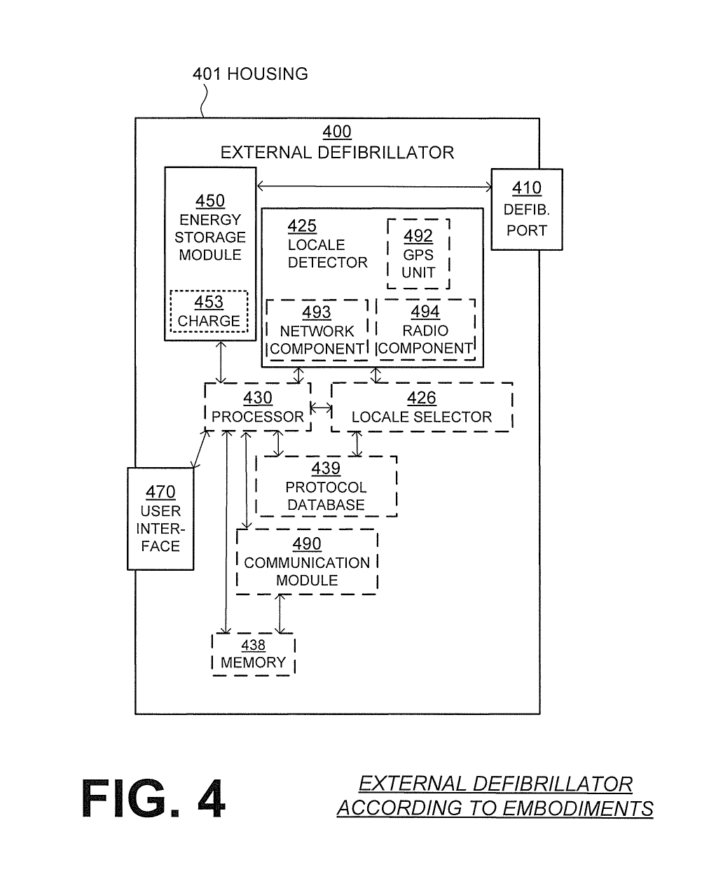

FIG. 4 is a functional block diagram showing components of another external defibrillator 400, such as the one shown in FIG. 1, which is made according to certain embodiments. Defibrillator 400 includes a housing 401, an energy storage module 450 within the housing 401 for storing an electrical charge 453, a defibrillation port 410 for guiding via electrodes the stored electrical charge 453 to a person, a processor 430, a memory 438, and a communication module 490. Defibrillator 400 also includes a user interface 470 structured to deliver prompts to a user during a defibrillation session.

Defibrillator 400 further includes a locale detector 425 in the housing 401 structured to determine a present locale of the defibrillator 400. The prompts delivered by the user interface 470 to a user during a defibrillation session are in accordance with a protocol that is selected based on the present locale. In certain embodiments, the processor 430 includes the locale detector 425. User interface 470 may be further structured to prompt the user to provide an indication of the present locale. In these embodiments, the locale detector 425 may be structured to determine the present locale based on the indication provided by the user.

Defibrillator 400 optionally includes a geographic positioning system (GPS) unit 492 within the housing 401 for providing GPS coordinates of the defibrillator 400. In these embodiments, the locale detector 425 may be structured to determine the present locale of the defibrillator 400 based on the GPS coordinates of the defibrillator 400. The locale detector 425 may include the GPS unit 492 or, alternatively, the GPS unit 492 may be integrated with or physically separate from the locale detector 425.

Defibrillator 400 optionally includes a network component 493 within the housing 401 for establishing a connection to a wireless network having a range, the present locale being within the range. In these embodiments, the locale detector 425 may be structured to determine the present locale of the defibrillator 400 based on the wireless network to which the network component 493 establishes the connection. In alternative embodiments, the network component 493 may be used for detecting a cellular network. In these embodiments, the locale detector 425 may be structured to determine the present locale of the device based on the cellular network detected by the network component. The locale detector 425 may include the network component 493 or, alternatively, the network component 493 may be integrated with or physically separate from the locale detector 425.

Defibrillator 400 optionally includes a radio component 494 within the housing 401 for detecting a localizable signal. In these embodiments, the locale detector 425 may be structured to determine the present locale of the defibrillator 400 based on the localizable signal detected by the radio component 494. The locale detector 425 may include the radio component 494 or, alternatively, the radio component 494 may be integrated with or physically separate from the locale detector 425.

In certain embodiments, the protocol specifies a particular language in which the user interface 470 is to deliver the prompts to the user during the defibrillation session. User interface 470 may be structured to deliver the prompts in accordance with a default protocol if the locale detector 425 is unable to determine the present locale. In embodiments where a present locale is determined by the locale detector 425, the user interface 470 may be further structured to prompt the user to provide a confirmation of the present locale.

Defibrillator 400 optionally includes a locale selector 426 in the housing 401 structured to select the protocol based on the present locale. In certain embodiments, the processor 430 may include the locale detector 425, the locale selector 426, or both the locale detector 425 and the locale selector 426.

In certain embodiments, the locale selector 426 is further structured to determine whether the selected protocol is supported by the defibrillator 400. Responsive to a determination that the selected protocol is not supported by the defibrillator 400, the user interface 470 may be structured to deliver the prompts in accordance with an initial protocol, e.g., a default protocol.

In certain embodiments, the user interface 470 is further structured to stop delivering the prompts in accordance with the protocol responsive to receiving an indication to stop using the protocol. User interface 470 may be structured to receive the indication to stop using the protocol from the user. Alternatively, the indication to stop using the protocol may be based on a determination that the present locale has changed or that the user is speaking a language that is different from an initial language. User interface 470 may be further structured to revert back to delivering the prompts in accordance with an initial protocol, to start delivering the prompts in accordance with a default protocol, or both. Also, the user interface 470 may be structured to not stop delivering the prompts in accordance with the protocol after a specified period of time has passed during a defibrillation session.

Defibrillator 400 optionally includes a protocol database 439 structured to store a listing of supported protocols. In these embodiments, the locale selector 426 may be structured to determine whether the selected protocol is within the listing of supported protocols. Database 439 may be within the housing 401 of the defibrillator 400. Alternatively or in addition thereto, the database 439 may be at a location that is remote from the defibrillator 400. In embodiments where the database 439 is located remote from the defibrillator 400, the communication module 490 within the housing 401 may establish a wireless connection to provide access to the database 439.

In certain embodiments, a record may be created from one of diagnosing and treating a person. In these embodiments, a code may be entered in the record that denotes the present locale. A time may be further determined, and a code may be entered in the record that denotes the determined time. In certain embodiments, a time zone is further detected from the present locale and the time is determined from the detected time zone.

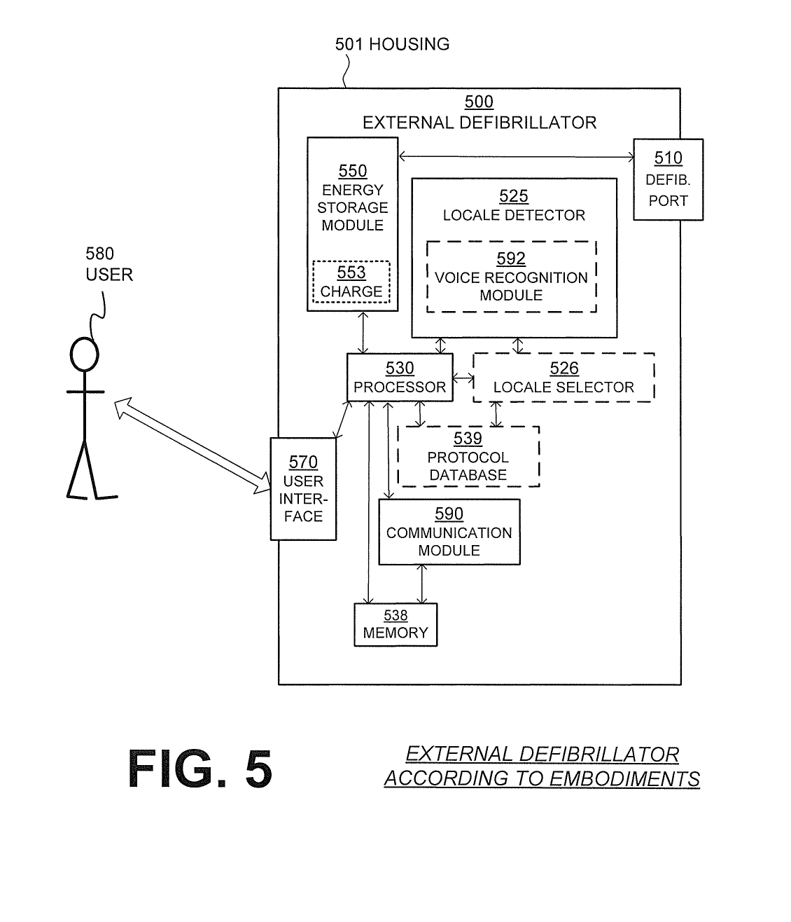

FIG. 5 is a functional block diagram showing components of another external defibrillator 500, such as the one shown in FIG. 1, which is made according to certain embodiments. Defibrillator 500 includes a housing 501, an energy storage module 550 for storing an electrical charge 553, a defibrillation port 510, a processor 530, a memory 538, a communication module 590, a locale detector 525, and a user interface 570 structured to interact with, e.g., provide prompts to, a user 580. Defibrillator 500 is similar to the defibrillator 400 of FIG. 4 in that the defibrillator 500 also has an optional locale selector 526 and optional protocol database 539. Locale detector 525 has an optional voice recognition module 592 structured to identify which language the user 580 is speaking and/or what the user 580 is saying, for example.

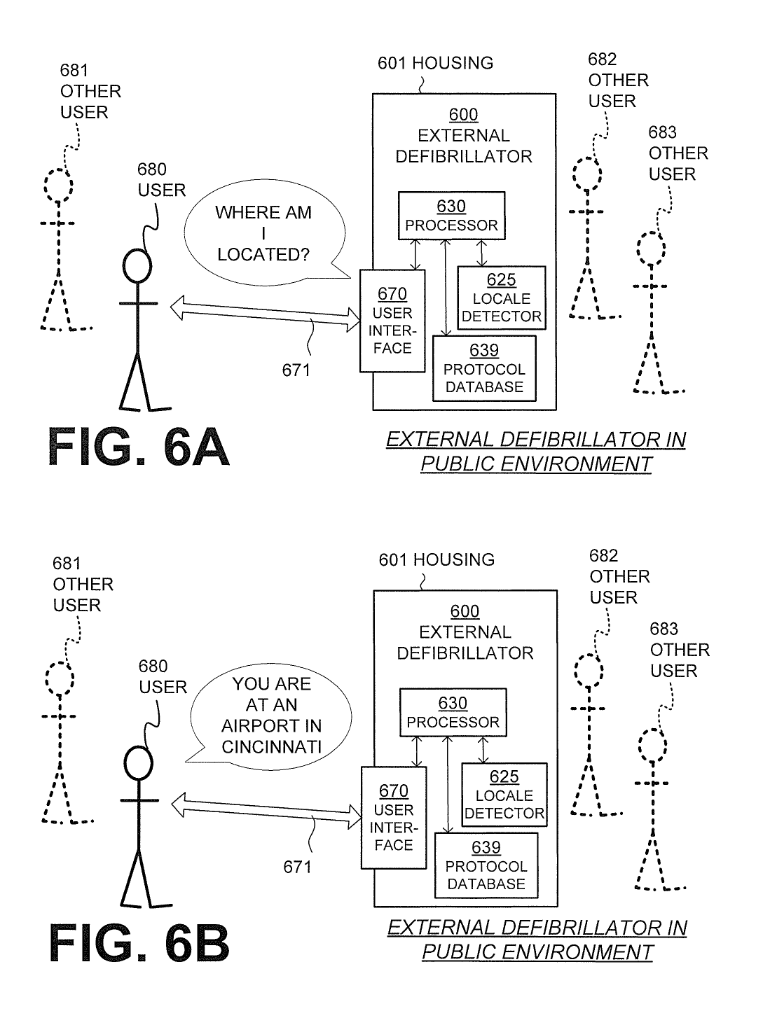

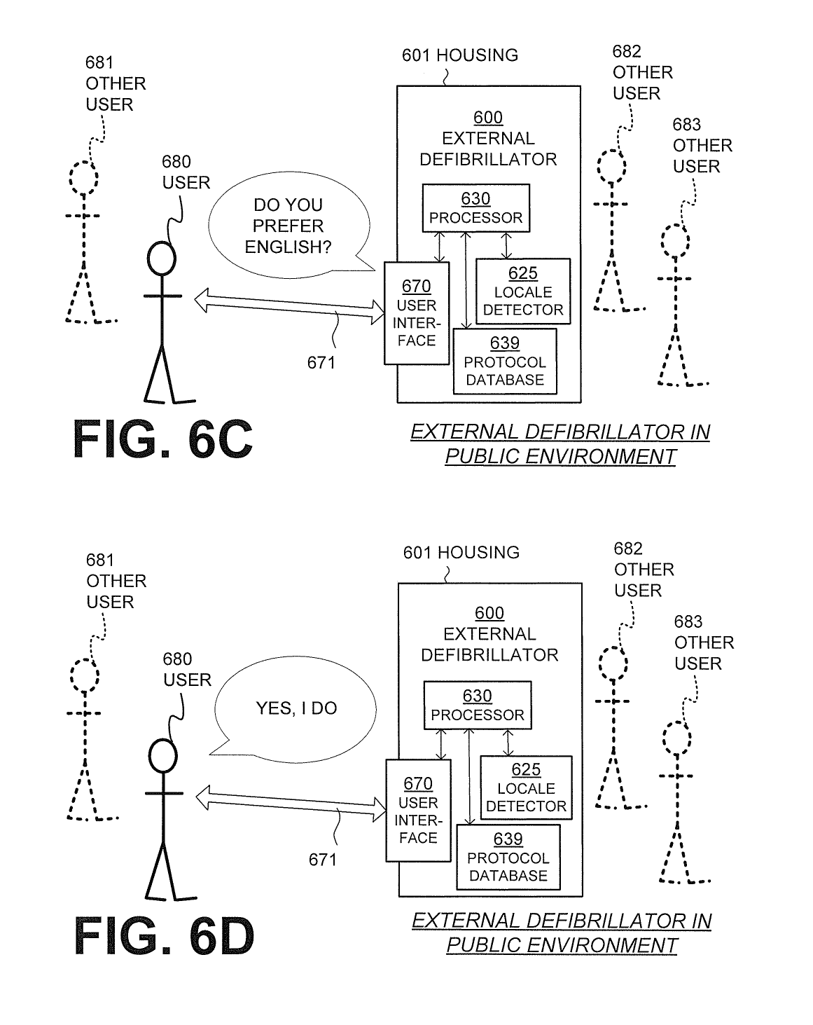

FIG. 6A is a diagram showing an external defibrillator 600 prompting a user 680 to provide an indication of the present locale according to embodiments. Defibrillator 600 includes a housing 601, a processor 630, a language detector 625, and a user interface 670 structured to interact with, e.g., provide prompts to or ask questions of, the user 680, as indicated generally by 671. There may be other users 681, 682, and 683 within the vicinity of the defibrillator 600 and the user 680. In the example, the defibrillator 600 is asking the user 680 to provide an indication of the present locale.

FIG. 6B is a diagram showing the user 680 of FIG. 6A providing an indication of the present locale to the external defibrillator 600 according to embodiments. In the example, the user 680 provides such an indication by stating that the defibrillator 600 is at an airport in Cincinnati.

FIG. 6C is a diagram showing the external defibrillator 600 of FIG. 6B prompting the user 680 to provide a confirmation of a particular language according to embodiments. Defibrillator 600 does this by asking the user whether he or she speaks a particularly language, e.g., English. Defibrillator 600 may select which language to present to the user 680 based on the determined present locale. For example, if the user 680 indicates that the defibrillator 600 is at an airport in the United States, the defibrillator 600 may select English as the language to present in the query to the user 680.

FIG. 6D is a diagram showing the user 680 of FIG. 6C providing a confirmation of the language to the external defibrillator 600 according to embodiments. The user 680 does so by answering the question in the affirmative, thus indicating that he or she prefers to speak English.

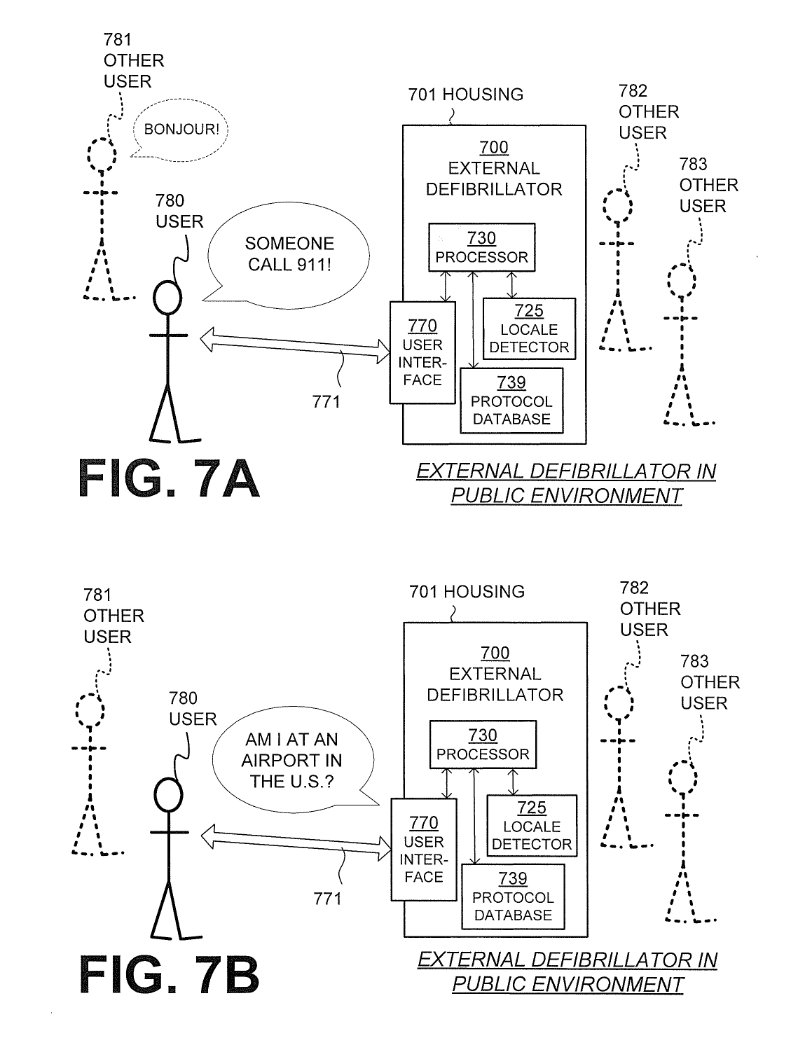

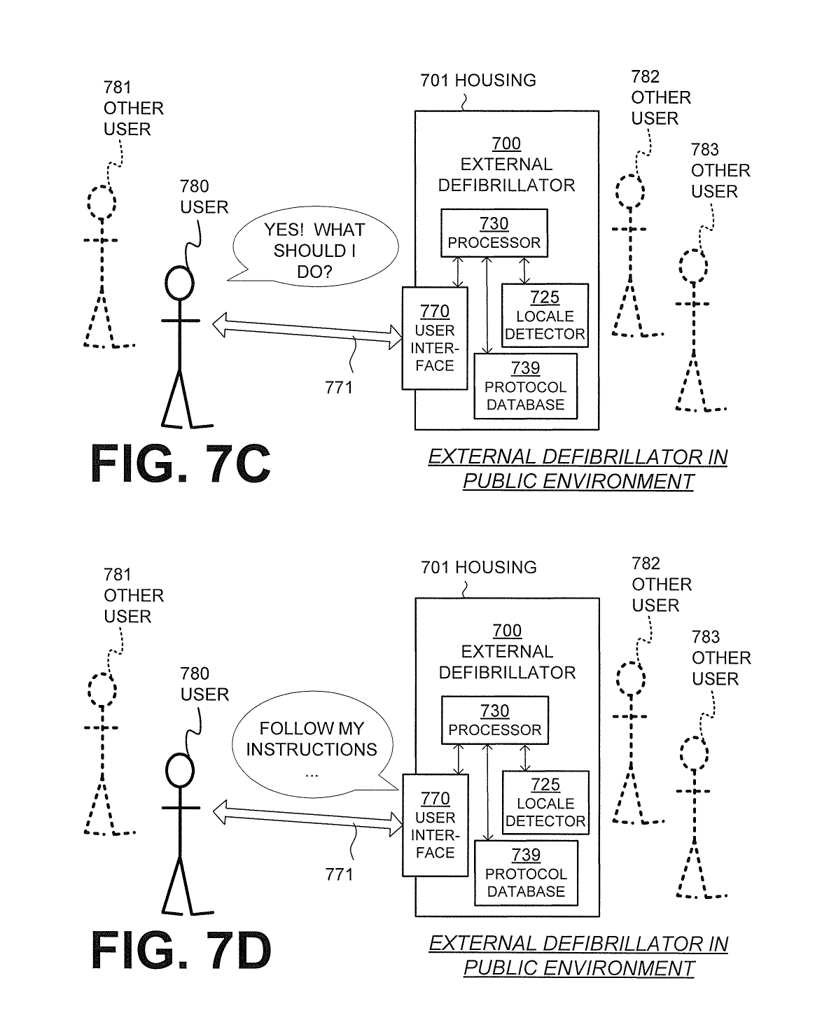

FIG. 7A is a diagram showing a user 780 crying out for help in the vicinity of an external defibrillator 700 according to embodiments. Defibrillator 700 includes a housing 701, a processor 730, a language detector 725, and a user interface 770 structured to interact with, e.g., provide prompts to, the user 780, as indicated generally by 771. There may be other users 781, 782, and 783 within the vicinity of the defibrillator 700 and the user 780. For example, the defibrillator 700 may be situated at a public location, such as an international airport, where the user 780 speaks one language and at least one of the other users 781 speaks a different language.

FIG. 7B is a diagram showing the external defibrillator 700 of FIG. 7A prompting the user 780 to provide a confirmation of a determined present locale according to embodiments. In the example, the locale detector 725 has made a determination that the present locale is an airport in the United States. This determination may be based on the user 780, who is situated closest to the defibrillator 700, crying out for help in English and another user 781 speaking in a different language, for example. Defibrillator 700 prompts the user 780 for confirmation of the determination by asking the user 780 whether the defibrillator 700 is indeed presently at an airport in the United States.

FIG. 7C is a diagram showing the user 780 of FIG. 7B providing a confirmation of the present locale to the external defibrillator 700 according to embodiments. The user 780 does so by answering the question in the affirmative, thus indicating that the present locale of the defibrillator 700 is indeed a U.S. airport in the example.

FIG. 7D is a diagram showing the external defibrillator 700 of FIG. 7C beginning to provide the user 780 with instructions for using the external defibrillator 700 according to embodiments. In the example, the user interface 770 of the defibrillator 700 is beginning to deliver prompts, e.g., voice prompts, to the user 780 based on the present locale, e.g. in a language that is particularly relevant to the present locale.

The functions of this description may be implemented by one or more devices that include logic circuitry. The device performs functions and/or methods as are described in this document. The logic circuitry may include a processor that may be programmable for a general purpose, or dedicated, such as microcontroller, a microprocessor, a Digital Signal Processor (DSP), etc. For example, the device may be a digital computer like device, such as a general-purpose computer selectively activated or reconfigured by a computer program stored in the computer. Alternately, the device may be implemented by an Application Specific Integrated Circuit (ASIC), etc.

Moreover, methods are described below. The methods and algorithms presented herein are not necessarily inherently associated with any particular computer or other apparatus. Rather, various general-purpose machines may be used with programs in accordance with the teachings herein, or it may prove more convenient to construct more specialized apparatus to perform the required method steps. The required structure for a variety of these machines will become apparent from this description.

In all cases there should be borne in mind the distinction between methods in this description, and the method of operating a computing machine. This description relates both to methods in general, and also to steps for operating a computer and for processing electrical or other physical signals to generate other desired physical signals.

Programs are additionally included in this description, as are methods of operation of the programs. A program is generally defined as a group of steps leading to a desired result, due to their nature and their sequence. A program is usually advantageously implemented as a program for a computing machine, such as a general-purpose computer, a special purpose computer, a microprocessor, etc.

Storage media are additionally included in this description. Such media, individually or in combination with others, have stored thereon instructions of a program made according to the invention. A storage medium according to the invention is a computer-readable medium, such as a memory, and is read by the computing machine mentioned above.

Performing the steps or instructions of a program requires physical manipulations of physical quantities. Usually, though not necessarily, these quantities may be transferred, combined, compared, and otherwise manipulated or processed according to the instructions, and they may also be stored in a computer-readable medium. These quantities include, for example electrical, magnetic, and electromagnetic signals, and also states of matter that can be queried by such signals. It is convenient at times, principally for reasons of common usage, to refer to these quantities as bits, data bits, samples, values, symbols, characters, images, terms, numbers, or the like. It should be borne in mind, however, that all of these and similar terms are associated with the appropriate physical quantities, and that these terms are merely convenient labels applied to these physical quantities, individually or in groups.

This detailed description is presented largely in terms of flowcharts, display images, algorithms, and symbolic representations of operations of data bits within at least one computer readable medium, such as a memory. Indeed, such descriptions and representations are the type of convenient labels used by those skilled in programming and/or the data processing arts to effectively convey the substance of their work to others skilled in the art. A person skilled in the art of programming may use these descriptions to readily generate specific instructions for implementing a program according to the present invention.

Often, for the sake of convenience only, it is preferred to implement and describe a program as various interconnected distinct software modules or features, individually and collectively also known as software. This is not necessary, however, and there may be cases where modules are equivalently aggregated into a single program with unclear boundaries. In any event, the software modules or features of this description may be implemented by themselves, or in combination with others. Even though it is said that the program may be stored in a computer-readable medium, it should be clear to a person skilled in the art that it need not be a single memory, or even a single machine. Various portions, modules or features of it may reside in separate memories, or even separate machines. The separate machines may be connected directly, or through a network, such as a local access network (LAN), or a global network, such as the Internet.

It will be appreciated that some of these methods may include software steps that may be performed by different modules of an overall software architecture. For example, data forwarding in a router may be performed in a data plane, which consults a local routing table. Collection of performance data may also be performed in a data plane. The performance data may be processed in a control plane, which accordingly may update the local routing table, in addition to neighboring ones. A person skilled in the art will discern which step is best performed in which plane.

An economy is achieved in the present document in that a single set of flowcharts is used to describe both programs, and also methods. So, while flowcharts are described in terms of boxes, they can mean both method and programs.

For this description, the methods may be implemented by machine operations. In other words, embodiments of programs are made such that they perform methods of the invention that are described in this document. These may be optionally performed in conjunction with one or more human operators performing some, but not all of them. As per the above, the users need not be collocated with each other, but each only with a machine that houses a portion of the program. Alternately, some of these machines may operate automatically, without users and/or independently from each other.

Methods are now described.

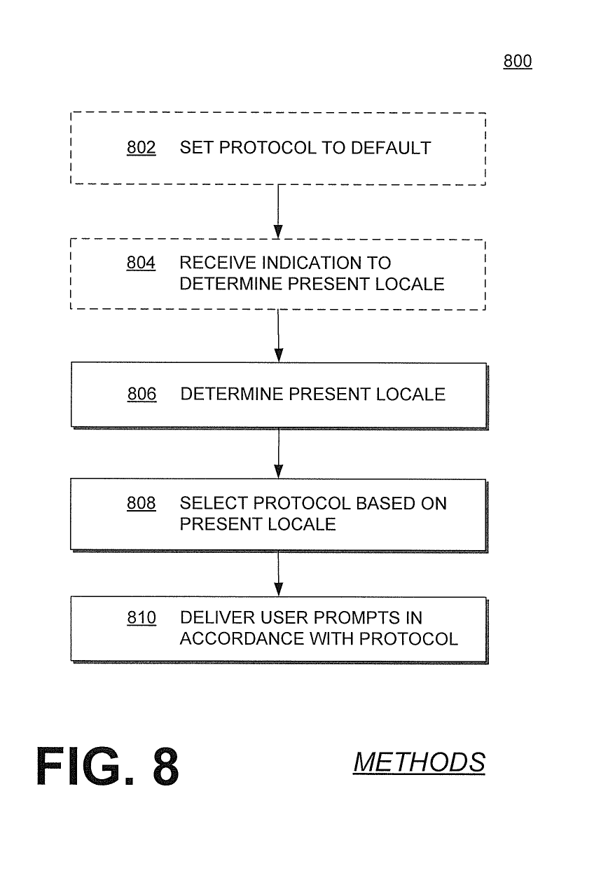

FIG. 8 is a flowchart 800 for illustrating methods of an external defibrillator delivering prompts in accordance with a selected protocol according to embodiments.

According to an optional operation at 802, the defibrillator sets the protocol to be used by the defibrillator to a default protocol. The default protocol may specify any of a number of specifications, parameters, etc. with regard to the defibrillator delivering prompts to a user during a defibrillation session, for example.

According to an optional operation at 804, the defibrillator receives an indication to determine the present locale. For example, a user may cause the defibrillator to initiate an internal update due to the defibrillator being moved from a first location to a second location having a protocol that is different from the protocol of the first location.

According to an operation at 806, the defibrillator determines a present locale of the defibrillator. For example, a locale detector may determine the present locale based on GPS coordinates of the device, a wireless network to which a network component of the device establishes a connection, a cellular network detected by the network component, a localizable signal detected by a radio component, an indication provided by a user, or any combination thereof.

According to an operation at 808, the defibrillator selects a protocol based on the present locale determined by the operation at 806. For example, the defibrillator may select a protocol based on the present locale being a certain airport within a particular city.

According to an operation at 810, the defibrillator delivers prompts to a user during a defibrillation session in accordance with the selected protocol. The defibrillator may deliver the prompts by way of a user interface, for example. The prompts may include audible prompts, visual prompts, or both audible prompts and visual prompts.

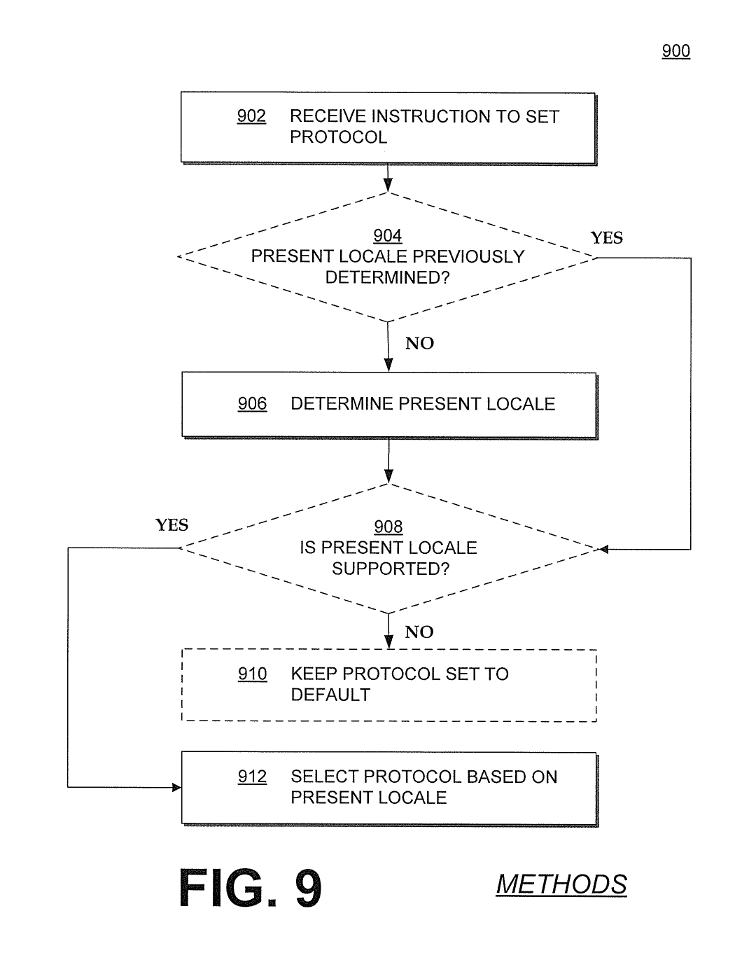

FIG. 9 is a flowchart 900 for illustrating methods of an external defibrillator setting a protocol based on a determination of the present locale according to embodiments.

According to an operation at 902, the defibrillator receives an instruction to set the protocol to be used by the defibrillator.

According to an optional operation at 904, a determination is made as to whether the present locale has been previously determined. If so, processing may continue to an optional operation at 908 or directly to an operation at 912; otherwise, processing continues to an operation at 906.

According to the operation at 906, the defibrillator determines the present locale of the defibrillator. For example, a locale detector of the defibrillator may determine the present locale based on GPS coordinates of the device, a wireless network to which a network component of the device establishes a connection, a cellular network detected by the network component, a localizable signal detected by a radio component, an indication provided by a user, or any combination thereof.

According to the optional operation at 908, a determination is made as to whether the present locale is supported. For example, a locale selector of the defibrillator may determine whether the protocol associated with the present local is within a listing of supported protocols. Responsive to a determination that the present local is supported, processing may continue to the operation at 912; otherwise, processing may continue to an optional operation at 910.

According to the optional operation at 910, the defibrillator sets the protocol to a default protocol. If the current protocol is a default protocol, then the defibrillator simply maintains the setting of the protocol to the default protocol.

According to the operation at 912, the defibrillator selects a protocol based on the determined present locale. For example, the defibrillator may select a protocol based on the present locale being a certain airport within a particular city.

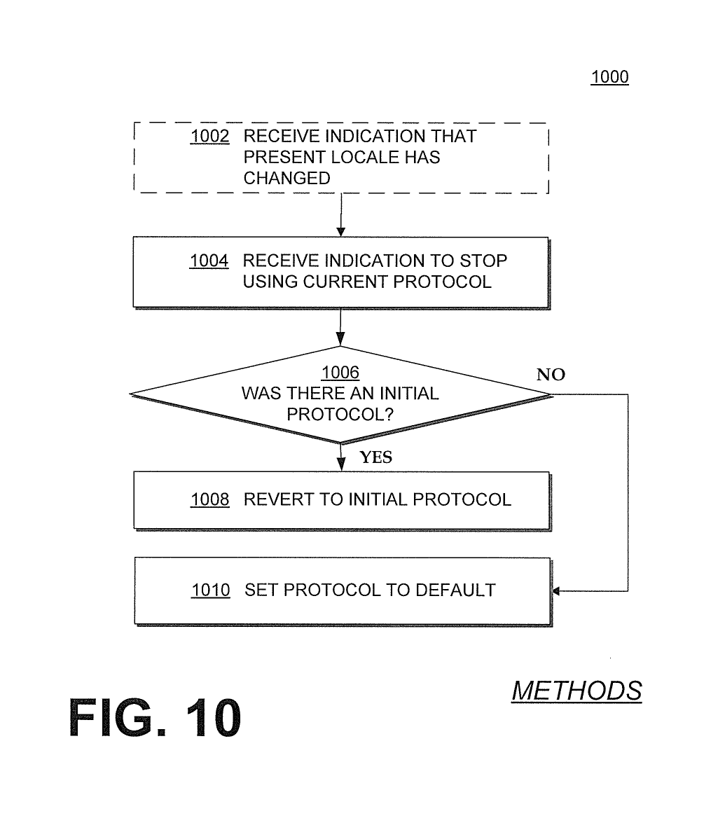

FIG. 10 is a flowchart 1000 for illustrating methods of an external defibrillator switching away from a current protocol for user prompts according to embodiments.

According to an optional operation at 1002, the defibrillator receives an indication that the present locale has changed. For example, the indication may be provided directly by a user. Alternatively or in addition thereto, the indication may be based on a determination by a GPS unit that the GPS coordinates of the device have changed, a network unit losing connection to a wireless network, the network unit no longer detecting a previously detected cellular network, a radio component no longer detecting a previously detected localizable signal, or any combination thereof.

According to an operation at 1004, the defibrillator receives an indication to stop using the current protocol. For example, the indication to stop using the protocol may be based on a determination that the user is speaking a language that is different from an initial language.

According to an operation at 1006, a determination is made as to whether there was an initial protocol. If so, processing may continue to an operation at 1008; otherwise, processing continues to an operation at 1010.

According to the operation at 1008, the defibrillator reverts to an initial protocol. For example, a user interface of the defibrillator may revert back to delivering prompts to a user during a defibrillation session in accordance with an initial protocol.

According to the operation at 1010, the defibrillator sets the protocol to a default protocol. For example, a user interface of the defibrillator may start delivering the prompts in accordance with a default protocol.

In this description, numerous details have been set forth in order to provide a thorough understanding. In other instances, well-known features have not been described in detail in order to not obscure unnecessarily the description.

A person skilled in the art will be able to practice the present invention in view of this description, which is to be taken as a whole. The specific embodiments as disclosed and illustrated herein are not to be considered in a limiting sense. Indeed, it should be readily apparent to those skilled in the art that what is described herein may be modified in numerous ways. Such ways can include equivalents to what is described herein. In addition, the invention may be practiced in combination with other systems.

The following claims define certain combinations and subcombinations of elements, features, steps, and/or functions, which are regarded as novel and non-obvious. Additional claims for other combinations and subcombinations may be presented in this or a related document.

What is claimed is:

1. An external medical device, comprising:
a housing;
an energy storage module within the housing for storing an electrical charge;
a defibrillation port for guiding via electrodes the stored electrical charge to a person;
a user interface structured to deliver prompts to a user during a defibrillation session; and
a locale detector in the housing structured to determine a present locale of the device,
in which the prompts delivered by the user interface to the user during the defibrillation session are in accordance with a protocol that is selected based on the present locale.

2. The device of claim 1, further comprising:
a processor, and
in which the processor includes the locale detector.

3. The device of claim 1, in which
the protocol specifies a particular language in which the user interface is to deliver the prompts to the user during the defibrillation session.

4. The device of claim 1, in which
the user interface is structured to deliver the prompts in accordance with a default protocol if the locale detector is unable to determine the present locale.

5. The device of claim 1, in which
the user interface is further structured to prompt the user to provide a confirmation of the present locale.

6. The device of claim 1, further comprising:
a geographic positioning system (GPS) unit within the housing for providing GPS coordinates of the device, and
in which the locale detector is structured to determine the present locale of the device based on the GPS coordinates of the device.

7. The device of claim 1, further comprising:
a network component within the housing for establishing a connection to a wireless network having a range, the present locale being within the range, and
in which the locale detector is structured to determine the present locale of the device based on the wireless network to which the network component establishes the connection.

8. The device of claim 1, further comprising:
a network component within the housing for detecting a cellular network, and
in which the locale detector is structured to determine the present locale of the device based on the cellular network detected by the network component.

9. The device of claim 1, further comprising:
a radio component within the housing for detecting a localizable signal, and
in which the locale detector is structured to determine the present locale of the device based on the localizable signal detected by the radio component.

10. The device of claim 1, in which
the user interface is further structured to prompt the user to provide an indication of the present locale, and
in which the locale detector is structured to determine the present locale based on the indication provided by the user.

11. The device of claim 1, further comprising:
a locale selector in the housing structured to select the protocol based on the present locale.

12. The device of claim 11, further comprising:
a processor, and
in which the processor includes the locale detector, the locale selector, or both the locale detector and the locale selector.

13. The device of claim 11, in which
the locale selector is further structured to determine whether the selected protocol is supported by the device.

14. The device of claim 13, in which
responsive to a determination that the selected protocol is not supported by the device, the user interface is structured to deliver the prompts in accordance with an initial protocol.

15. The device of claim 14, in which
the initial protocol is a default protocol.

16. The device of claim 11, further comprising:
a database structured to store a listing of supported protocols, and
in which the locale selector is structured to determine whether the selected protocol is within the listing of supported protocols.

17. The device of claim 16, in which the database is within the housing.

18. The device of claim 16, further comprising:
a communication module within the housing to establish a wireless connection to provide access to the database, and
in which the database is at a location that is remote from the device.

19. The device of claim 1, in which
the user interface is further structured to stop delivering the prompts in accordance with the protocol responsive to receiving an indication to stop using the protocol.

20. The device of claim 19, in which
the user interface is structured to receive the indication to stop using the protocol from the user.

21. The device of claim 19, in which
the indication to stop using the protocol is based on a determination that the present locale has changed.

22. The device of claim 19, in which
the indication to stop using the protocol is based on a determination that the user is speaking a language that is different from an initial language.

23. The device of claim 19, in which
the user interface is further structured to revert back to delivering the prompts in accordance with an initial protocol.

24. The device of claim 19, in which
the user interface is further structured to start delivering the prompts in accordance with a default protocol.

25. The device of claim 1, in which
the user interface is structured to not stop delivering the prompts in accordance with the protocol after a specified period of time has passed during the defibrillation session.

26. The device of claim 1, in which
a record is created from one of diagnosing and treating the person, and
a code is entered in the record that denotes the present locale.

27. The device of claim 26, in which
a time is further determined, and
a code is entered in the record that denotes the determined time.

28. The device of claim 27, in which
a time zone is further detected from the present locale, and
the time is determined from the detected time zone.

29. A method in an external defibrillator, the method comprising:
determining a present locale of the defibrillator;
selecting a protocol based on the determined present locale; and
delivering by way of a user interface of the external defibrillator one or more prompts to a user during a defibrillation session in accordance with the selected protocol.

30. The method of claim 29, in which
the prompts include audible prompts, visual prompts, or both audible prompts and visual prompts.

31. The method of claim 29, further comprising:
setting the protocol to a default protocol prior to the determining.

32. The method of claim 29, further comprising:
receiving an indication to determine the present locale prior to the determining.

33. The method of claim 29, further comprising:
determining whether the present locale is supported.

34. The method of claim 29, further comprising:
receiving an indication to stop using the selected protocol.

35. The method of claim 34, further comprising:
determining whether there was an initial protocol.

36. The method of claim 35, further comprising:
responsive to a determination that there was an initial protocol, delivering the prompts to the user in accordance with the initial protocol.

37. The method of claim 35, further comprising:
responsive to a determination that there was not an initial protocol, delivering the prompts to the user in accordance with a default protocol.
